SEQUENCE LISTING (4 sequences)

SEQ ID NO: 1 - protein, 9 aa, Homo sapiens
EEQYNSTYR

SEQ ID NO: 2 - protein, 25 aa, Homo sapiens
EEQFNSTFCVVSVLTVVHQDWLNGK

SEQ ID NO: 3 - protein, 9 aa, Homo sapiens
EEQYNSTFR

SEQ ID NO: 4 - protein, 9 aa, Homo sapiens
EEQFNSTYR

(19) United States
(12) Patent Application Publication
(10) Pub. No.: US 2013/0058917 A1
(43) Pub. Date: Mar. 7, 2013

(54) ANTIBODY COMPOSITION OBTAINED BY FRACTIONATION OF PLASMA IMMUNOGLOBULINS AFFINITY CHROMATOGRAPHY ON A SAMBUCUS NIGRA AFFINITY COLUMN

(71) Applicants: Fabian Käsermann, Burgdorf (CH); Sylvia Miescher, Bern (CH)

(72) Inventors: Fabian Käsermann, Burgdorf (CH); Sylvia Miescher, Bern (CH)

(21) Appl. No.: 13/696,483

(22) PCT Filed: May 4, 2011
(86) PCT No.: PCT/EP2011/057097

§ 371 Date: Nov. 13, 2012

(30) Foreign Application Priority Data
May 7, 2010 (EP) 10004845.3

(51) Int. Cl. C07K 1/22; A61K 39/395; A61P 29/00; A61P 25/28; A61P 25/00; A61P 19/02; A61P 7/00; A61P 17/00; A61P 25/16; C07K 16/00; A61P 37/06

(52) U.S. Cl. 424/130.1; 530/387.1

(57) ABSTRACT

The invention relates to populations of antibodies obtainable by fractionation of plasma immunoglobulins, in particular plasma IgG, by affinity chromatography on a Sambucus nigra affinity column, and uses thereof.

This application is the United States national stage of PCT/EP2011/057097, filed May 4, 2011, (published as WO 2011/138354), and also claims priority to European Patent Application No. 10 004 845.3, filed May 7, 2010, both of which are incorporated herein by reference.

The invention relates to populations of antibodies obtainable by fractionation of plasma immunoglobulins, in particular plasma IgG, by affinity chromatography on a Sambucus nigra affinity column, and uses thereof.

### SEQUENCE LISTING

The instant application contains a Sequence Listing which has been submitted in ASCII format via EFS-Web and is hereby incorporated by reference in its entirety. Said ASCII copy, created on Nov. 5, 2012, is named WO_A170.5T25.txt and is 943 bytes in size.

### BACKGROUND TO THE INVENTION

Immunoglobulins, also known as antibodies, are the major secretory products of the immune system. They are typically formed of basic structural units—each with 2 heavy chains and 2 light chains—to form monomers with one such unit, dimers with two units, pentamers with five units, or hexamers with six units. Antibodies play a significant role in innate immunity. In a natural immune response to a pathogen, complexes are formed between the pathogen and antibodies. These immune complexes activate a wide range of effector functions, thus leading to the killing, removal and destruction of the pathogen. Antibodies can also react with the body's own antigens, which can lead to autoimmune diseases, and contribute to chronic inflammatory symptoms. Antibodies can have an anti-inflammatory activity, for example by targeting and neutralising various mediators in the inflammatory cascade.

There are five major isotypes of immunoglobulins, which perform different roles. IgG provides the majority of antibody-based immunity against invading pathogens, and is discussed in more detail below. IgM occurs in a membrane-bound form and in solution. In solution, it typically forms a pentamer, providing high avidity in binding to the antigen. It is often the first, immediate defence against infections before sufficient specific IgG is produced. IgA usually occurs as a dimer and is found in mucosal areas, e.g. the gut, lung and urogenital tract. It protects these surfaces against colonization by pathogens. IgE is mainly involved in allergic reactions. It binds to allergens and triggers histamine release from mast cells and basophils. IgD is found mainly on the surface of B lymphocytes that have not been exposed to antigens.

In humans, four subclasses of IgGs are defined and numbered according to their relative concentrations in normal serum: IgG1, IgG2, IgG3 and IgG4, which respectively account for approximately 60%, 25%, 10% and 5% of serum IgG, each IgG subclass possessing unique effector functions.

In general, each individual monomeric immunoglobulin unit, e.g. an IgG molecule, consists of two identical light chains and two identical heavy chains, which in turn comprise repeating structural motifs of approximately 110 amino acid residues. Domains of the light and heavy chains pair in covalent and non-covalent associations, thus forming three independent protein moieties connected through a flexible linker, the so-called hinge region. Two of these moieties are referred to as Fab (antigen-binding fragment) regions and are of identical structure. Each of the Fab regions forms the same specific antigen-binding site. The third moiety is the Fc (crystallizable fragment) region, which forms interaction sites for ligands that activate clearance and transport mechanisms.

IgGs play an important role in diseases. IgG1-type antibodies are the most commonly used antibodies in cancer immunotherapy where antibody-dependent cell-mediated cytotoxicity (ADCC) is often deemed important. Furthermore, IgGs are known to mediate both pro- and anti-inflammatory activities through interactions mediated by their Fc fragments. On one hand, interactions between Fc and its respective receptors are responsible for the pro-inflammatory properties of immune complexes and cytotoxic antibodies. On the other hand, intravenous gamma globulin (IVIG) and its Fc fragments are anti-inflammatory and are widely used to suppress inflammatory diseases. The precise mechanism of such paradoxical properties is unclear but it has been proposed that glycosylation of IgG is crucial for regulation of cytotoxicity and inflammatory potential of IgG.

To date, research into the role of glycosylation in the regulation of cytotoxicity and the inflammatory effects mediated by IgGs has mainly focused on the Fc region. Glycosylation of IgG is essential for binding to all FcyRs by maintaining an open conformation of the two heavy chains. This requirement of IgG glycosylation for FcyR binding explains the inability of de-glycosylated IgG antibodies to mediate in vivo triggered inflammatory responses, such as antibody-dependent cell-mediated cytotoxicity (ADCC), phagocytosis and the release of inflammatory mediators. A link between autoimmune states and specific glycosylation patterns of IgG antibodies has been observed in patients with rheumatoid arthritis and several autoimmune vasculities in which decreased galactosylation and sialylation of IgG antibodies have been reported (Parekh et al., Nature 316, 452 (1985); Rademacher et al., Proc. Natl. Acad. Sci. USA 91, 6123 (1994); Matsumoto et al., 128, 621 (2000)).

International patent application WO 2007/117505 discloses a polypeptide containing at least one IgG Fc region, said polypeptide having a higher anti-inflammatory activity and a lower cytotoxic activity as compared to an unpurified antibody. In this application it was found that an IgG preparation enriched for increased sialylation of the N-linked glycosylation site on the Fc fragment showed an enhanced anti-inflammatory activity in vivo. In contrast, it was concluded that Fab fragments displayed no anti-inflammatory activity in this in vivo assay.

The international application WO 2008/057634 by the same inventors as WO 2007/117505 discloses a polypeptide containing at least one IgG Fc region, wherein said at least one IgG Fc region is glycosylated with at least one galactose moiety connected to a respective terminal sialic acid moiety by a α-2,6 linkage. The polypeptide of WO 2008/057634 has a higher anti-inflammatory activity as compared to an unpurified antibody. Also in this application it was found that an IgG preparation enriched for increased sialylation of the N-linked glycosylation site on the Fc fragment showed an enhanced anti-inflammatory activity in vivo. In contrast, it was concluded that Fab fragments displayed no anti-inflammatory activity in this in vivo assay. Further evidence that the anti-inflammatory activity is due to increased sialylation on the Fc fragment, rather than Fab fragment, is provided in international application WO 2009/079382 by the same inventors.

In a commentary by Nimmerjahn & Ravetch (JEM 204, 11-15 (2007)), the authors clearly teach that the sialylation in the Fc region is the key to the anti-inflammatory activity of IVIG. In addition they state that a generalized role for the antigen binding domain, located in the Fab regions, in the anti-inflammatory activity of IgG is unlikely, given that in their hands, intact IVIG and its Fc fragments have equivalent anti-inflammatory activity.

The international application WO 2007/005786 is directed to methods for controlling the properties of an Fc-containing molecule, said methods comprising altering the sialylation of the oligosaccharides in the Fc region. In this application it was found that the level of sialylation of the Fc oligosaccharides alters the affinity of recombinantly-produced therapeutic antibodies for Fcy receptors, resulting in modulation of various aspects of the biological actions of these antibodies. It was further discovered that the removal of sialic acid from the Fc oligosaccharides enhances the avidity of recombinantly-produced therapeutic antibodies for their target molecule. However, the effect is believed to be entirely Fc mediated, as no differences in the intrinsic affinity between each Fab arm and the target were observed. The inventors of WO 2007/005786 hypothesize that the removal of the charged static group from the Fc oligosaccharide allows for more flexibility in the overall antibody structure, thus providing a higher potential of interaction for the two binding domains in relationship of one to the other.

A recent review article by Jefferis (Jefferis, R.; Nat Rev Drug Discov. 2009 March; 8(3):226-34) discusses glycosylation as a strategy to improve antibody-based therapeutics. According to Jefferis, approx. 30% of polyclonal human IgG molecules bear N-linked oligosaccharides in the IgG Fab region and a higher level of galactosylation and sialylation for IgG Fab than for IgG Fc is observed. When present, the glycosylation is attached to the variable regions, and the functional significance of IgG Fab glycosylation of polyclonal IgG is not clear. Based on studies on monoclonal antibodies it is speculated that the glycosylation in the Fab region of an antibody can have a neutral, positive or negative influence on antigen binding. No incentive is given to study this further, nor to enrich the antibody population that is glycosylated in the Fab region.

Thus, antibody preparations having an increased amount of sialylation in the Fc region of antibodies have been intensely studied for the prevention and treatment of various diseases and also a potential role of glycosylation of the Fab region of antibodies has been discussed.

In International patent application PCT/US2010/028889 we reported the fractionation of antibodies by Sambucus nigra affinity chromatography, whereby all bound material was eluted with acidic lactose in a single fraction (+SA). We showed that the eluted fraction, which has an increased sialylation in the Fab region, has surprising immunomodulatory effects.

However, whereas a number of antibody products are currently in use for therapy, there is still a need to provide alternative and/or improved antibody preparations having a more beneficial effectiveness in clinical applications.

The solution to this technical problem is achieved by providing the embodiments characterised in the claims. Here, we disclose the further separation of the material bound to the Sambucus nigra affinity column, eluting first with a carbohydrate at neutral pH, followed by elution with a carbohydrate at acidic pH. We show that the antibodies eluted with acidic carbohydrate after pre-elution with neutral carbohydrate, have surprisingly enhanced immunomodulatory activity.

### SUMMARY OF THE INVENTION

One aspect of the invention is a method for producing an antibody population with enhanced immunomodulatory activity, including the following steps:
a. Subjecting an antibody preparation to affinity chromatography on Sambucus nigra agglutinin or an equivalent thereof,
b. Optionally collecting the non-binding antibody population (−SNA fraction),
c. Optionally washing the affinity matrix;
d. Eluting the binding population of antibodies with a carbohydrate at neutral pH (E1 fraction),
e. Eluting the remaining bound population of antibodies with a carbohydrate at acidic pH (E2 fraction),

Wherein the E2 fraction has an enhanced immunomodulatory activity when compared to the total eluate of the SNA matrix (+SNA fraction).

Preferably, the carbohydrate is a sugar, more preferably, the sugar is lactose.

Preferably, the neutral pH is a pH in the range of 6 to 8, more preferably in the range of 6.5 to 7.5, even more preferably, the pH is about 7.5.

Preferably, the acidic pH is a pH below 5, more preferably a pH below 4, even more preferably a pH below 3.5, most preferably a pH of about 3.

The antibody preparation is preferably an IgG preparation isolated from human plasma, more preferably an IgG preparation isolated from pooled human plasma from at least 1000 donors, even more preferably the antibody preparation is an IVIG or SCIG preparation.

The immunomodulatory activity may be determined using an in vitro assay for measuring anti-inflammatory activity. Preferably, the assay measures the inhibition of the effects of inflammatory stimuli on blood cells or cell lines, for example stimulation by phytohaemagglutinin, interferon-gamma, lipopolysaccharides (LPS), TLR agonists or other agents on peripheral blood mononuclear cells (PBMCs) or cell lines such as U937 or similar cell lines. The inflammatory effects can be determined, for example, by measuring cell surface markers or production of cytokines. Preferably, the immunomodulatory activity is determined by measuring the inhibition of phytohemagglutinin-induced or LPS_induced CD54 expression by monocytes or cytokine secretion of blood cells.

Preferably, the immunomodulatory activity of the antibody population obtainable in step e (E2 fraction) is at least 10% greater than the immunomodulatory activity of the total bound and eluted fraction (+SNA fraction) or the antibody population obtainable in step d (E1 fraction).

Another aspect of the invention is a population of antibodies obtainable by the methods described above.

Preferably, the population of antibodies obtainable by the elution with carbohydrate at neutral pH in step d of the method as outlined above (E1 fraction) has
a. about equivalent sialylation in the Fc region as the antibody preparation prior to affinity chromatography; and/or
b. at least 50%, preferably at least 60%, even more preferably at least 70%, even more preferably at least 80%, most preferably more than 90% higher sialylation of total glycans in the Fab region than the antibody preparation prior to affinity chromatography.

Preferably, the population of antibodies obtainable by the elution with carbohydrate at acidic pH in step e of the method as outlined above (E2 fraction) has
a. At least 15%, preferably at least 20%, even more preferably at least 22, 24, 25, 26, 27, 28, 29, 30% higher sialylation in the Fc region than the antibody preparation prior to affinity chromatography or the fraction of step d; and/or
b. At least 50%, preferably at least 60%, even more preferably at least 70%, even more preferably at least 80%, most preferably more than 90% higher sialylation of total glycans in the Fab region than the antibody preparation prior to affinity chromatography; and/or
c. At least 4%, preferably at least 5%, even more preferably at least 6% higher sialylation of total glycans in the Fab region than the antibody population of step d.

A further aspect of the invention is a pharmaceutical composition comprising an antibody population of the invention, and a pharmaceutically acceptable carrier or excipient.

A further aspect of the invention is an antibody population of the invention for use as a medicament.

Yet a further aspect of the invention is an antibody population of the invention for use in the treatment or prevention of an inflammatory condition. Preferably, the inflammatory condition is an autoimmune disease or a neurodegenerative disease. More preferably, the autoimmune or neurodegenerative disease is selected from Rheumatoid arthritis, Systemic Lupus Erythematosus (SLE), Antiphospholipid syndrome, immune thrombocytopenia (ITP), Kawasaki disease, Guillain Barré syndrome (GBS), multiple sclerosis (MS), chronic inflammatory demyelinating polyneuropathy (CIDP), skin blistering diseases, Dermatomyositis, Polymyositis, Alzheimer's Disease, Parkinson's Disease, Alzheimer's Disease related to Downs Syndrome, cerebral amyloid angiopathy, Dementia with Lewy bodies, Fronto-temporal lobar degeneration and vascular dementia.

### DETAILED DESCRIPTION OF THE INVENTION

The present invention relates to methods for producing populations of antibodies, as well as the resulting populations of antibodies, with enhanced immunomodulatory activity. The methods are based on a fractionation by affinity chromatography on a Sambucus nigra agglutinin (SNA) affinity matrix or equivalent thereof. The population of antibodies has an enhanced immunomodulatory activity when compared to the antibody preparation prior to fractionation or even when compared to the total fraction of antibodies eluted from the affinity matrix (+SNA). The immunomodulatory activity may be determined using, for example, an in vitro assay for measuring anti-inflammatory activity. The skilled person will be well aware of such assays. Preferably, the assay measures the inhibition of the effects of inflammatory stimuli on blood cells or cell lines, for example stimulation by phytohaemagglutinin, LPS; interferon-gamma, TLR agonists or other agents on peripheral blood mononuclear cells (PBMCs) or cell lines such as U937 or similar cell lines. The inflammatory effects can be determined, for example, by measuring cell surface markers or production of cytokines. Preferably, the immunomodulatory activity is determined by measuring the inhibition of phytohemagglutinin-induced or LPS-induced CD54 expression by monocytes.

The immunomodulatory activity of the population of antibodies is enhanced by at least 10%, preferably by at least 12%, 13% 14%, or 15%, more preferably by at least 18%, even more preferably by at least 20%, even more preferably by 21, 22, 23, 24, 25, 26, 27, 28, 29, 30%, most preferably by more than 30% (when compared to total +SNA IVIG).

Another embodiment of the invention is a population of antibodies, obtainable from an antibody preparation by the following steps:
a. Subjecting the antibody preparation to affinity chromatography on a Sambucus nigra agglutinin matrix or equivalent thereof,
b. Optionally collecting the non-binding population (−SNA fraction),
c. Optionally including a washing step, and
d. Eluting the binding population with a carbohydrate at neutral pH (E1 fraction), and
e. Eluting the still bound population with a carbohydrate at acidic pH (E2 fraction),

Wherein the E2 fraction has an enhanced immunomodulatory activity when compared to a total eluate of the SNA column (+SNA fraction).

A further embodiment of the invention is a method for producing an antibody population with enhanced immunomodulatory activity, including the following steps:
a. Subjecting an antibody preparation to affinity chromatography on Sambucus nigra agglutinin or an equivalent thereof,
b. Optionally collecting the non-binding population (−SNA fraction),
c. Optionally including a washing step,
d. Eluting the binding population with a carbohydrate at neutral pH (E1 fraction),
e. Eluting the remaining bound population with a carbohydrate at acidic pH (E2 fraction),

Wherein the E2 fraction has an enhanced immunomodulatory activity when compared to the total eluate of the SNA column (+SNA fraction).

The carbohydrate used for the elution is preferably a sugar, more preferably it is lactose or sialolactose, most preferred is lactose.

The antibody preparation is preferably isolated from human plasma, preferably human plasma pooled from at least 1000 donors, more preferably the antibody preparation is enriched in IgG, even more preferably, it is purified IgG. Most preferably, it is a preparation of human IgG formulated for intravenous or subcutaneous administration to patients, such as IVIg or SCIg, in particular therapeutic products such as Sandoglobulin, Privigen, or Hizentra. Typically the IgA and IgM content of the antibody preparation will be below 2%, preferably below 1.5%, more preferably below 1%.

The present invention also relates to a composition comprising a population of antibodies of the invention.

The term “composition” in accordance with the present invention relates to a composition which comprises an antibody population of the invention. As an antibody population of the invention is obtainable by fractionation on an affinity matrix which binds sialic acid residues, it is also a population of antibodies enriched from an antibody preparation, wherein the enriched population of antibodies has an altered sialylation pattern in the Fab region of the antibodies as compared to the antibody preparation prior to enrichment or which comprises at least a population of antibodies, wherein the population of antibodies has an altered sialylation pattern in the Fab region of the antibodies as compared to the antibody preparation prior to enrichment. The composition may, optionally, comprise further molecules capable of altering the characteristics of the population of antibodies of the invention thereby, for example, reducing, stabilizing, delaying, modulating and/or activating the function of the antibodies. The composition may be in solid, or liquid form and may be, inter alia, in the form of (a) powder(s), (a) tablet(s), (a) solution(s) or (an) aerosol(s).

In a preferred embodiment, the composition of the invention is a pharmaceutical composition optionally further comprising a pharmaceutically acceptable carrier, excipient and/or diluent.

In accordance with the present invention, the term “pharmaceutical composition” relates to a composition for administration to a patient, preferably a human patient. The pharmaceutical composition of the invention comprises a population of antibodies, recited above. The pharmaceutical composition of the present invention may, optionally and additionally, comprise a pharmaceutically acceptable carrier. By “pharmaceutically acceptable carrier” is meant a non-toxic solid, semisolid or liquid filler, diluent, encapsulating material or formulation auxiliary of any type. Examples of suitable pharmaceutical carriers are well known in the art and include sodium chloride solutions, phosphate buffered sodium chloride solutions, water, emulsions, such as oil/water emulsions, various types of wetting agents, sterile solutions, organic solvents etc. Preferably the carrier is a parenteral carrier, more preferably a solution that is isotonic with the blood of the recipient. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or further immunoglobulins; hydrophilic polymers such as polyvinylpyrrolidone; amino acids, such as glycine, proline, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

Compositions comprising such carriers can be formulated by well known conventional methods. Generally, the formulations are prepared by contacting the components of the pharmaceutical composition uniformly and intimately with liquid carriers or finely divided solid carriers or both. Then, if necessary, the product is shaped into the desired formulation.

These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgment of the ordinary clinician or physician. The pharmaceutical composition may be for administration once or for a regular administration over a prolonged period of time. Generally, the administration of the pharmaceutical composition should be in the range of for example 10 μg/kg of body weight to 2 g/kg of body weight for a single dose. However, a more preferred dosage might be in the range of 100 μg/kg to 1.5 g/kg of body weight, even more preferably 1 mg/kg to 1 g/kg of body weight and even more preferably 10 mg/kg to 500 mg/kg of body weight for a single dose. Administration of pharmaceutical compositions of the invention may be effected by different ways, e.g., by intravenous, intraperitoneal, subcutaneous, intramuscular, intradermal, oral, intranasal or intrabronchial administration.

The components of the pharmaceutical composition to be used for therapeutic administration must be sterile. Sterility is readily accomplished by filtration through sterile filtration membranes (e.g., 0.2 micron membranes).

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection. Preservatives and other additives may also be present such as, for example, antimicrobials, anti oxidants, chelating agents, and inert gases and the like. The pharmaceutical composition may comprise further agents depending on the intended use of the pharmaceutical composition.

Another embodiment is the medical use of an antibody population of the invention, or a composition, preferably a pharmaceutical composition comprising the antibody population of the invention.

Also included in the invention is the antibody population of the invention for use in the treatment or prevention of inflammatory conditions, for example autoimmune diseases and/or neurodegenerative diseases, such as Rheumatoid arthritis, Systemic Lupus Erythematosus (SLE), Antiphospholipid syndrome, immune thrombocytopenia (ITP), Kawasaki disease, Guillain Barré syndrome (GBS), multiple sclerosis (MS), chronic inflammatory demyelinating polyneuropathy (CIDP), skin blistering diseases, Dermatomyositis, Polymyositis, Alzheimer's Disease, Parkinson's Disease, Alzheimer's Disease related to Downs Syndrome, cerebral amyloid angiopathy, Dementia with Lewy bodies, Fronto-temporal lobar degeneration or vascular dementia. Preferably, the population of antibodies for use in these conditions has an increased sialic acid content, preferably the increased sialic acid content is in the Fab or the Fc region of the antibodies, more preferably in the Fab region.

The present invention also relates to the population of antibodies of the invention for use in the prevention and/or treatment of atherosclerosis, cancer and infections such as bacterial, viral or fungal infections.

It is particularly preferred that the population of antibodies of the invention may be used in the prevention and/or treatment of infections not amenable to traditional treatment regimens, for example due the occurrence of resistance to antibiotics.

All of the diseases described herein are well known to the skilled person and are defined in accordance with the prior art and the common general knowledge of the skilled person.

As described above, a population of antibodies of the invention is obtainable by fractionation of an antibody preparation on a Sambucus nigra affinity matrix. Sambucus nigra agglutinin is a lectin that is specific for terminal alpha 2,6-linked sialic acid residues. It is therefore likely that the binding of the population of antibodies to the affinity column is linked to enhanced sialylation of the antibody population of the invention as compared to the antibody population that is found in the flow-through of the affinity column and even the antibody population that is eluted with carbohydrate at neutral pH. The enhanced sialylation is likely to be found in the Fab region, but enhanced sialylation may also be seen in the Fc region. The terms “amount of sialylation” and “amount of sialic acid” are used interchangeably herein. As defined below, the altered amount of sialylation may be either an increase or a decrease in the amount of sialylation in the Fab region or in the Fc region of the antibodies. Preferably, the population of antibodies of the invention differs from the antibody preparation prior to affinity chromatography by having an amount of sialylation that is at least 1.25 times higher or lower, more preferably at least 1.5 times higher or lower, more preferably at least two times higher or lower such as at least three times or at least four times higher or lower as compared to the amount of sialylation in the Fab region in the antibody preparation prior to affinity chromatography. More preferably, the amount of sialylation is at least five times higher or lower as compared to the amount of sialylation in the Fab region in the antibody preparation prior to affinity chromatography. The term “times higher or lower” refers to a relative increase or decrease compared to the starting material when measuring % sialylated glycans of the total glycans. For example, if the antibody population prior to affinity chromatography has an amount of sialic acid of 15% of the glycans, then a population of antibodies having a three times higher or lower amount of sialic acid will have 45% or 5% glycans with sialic acid, respectively. In the case of IgG, the sialylation of Fc fragments is determined by digestion with a protease such as trypsin, followed by analysis of the resulting Fc-specific glycopeptides (for example as described in Example 2, method (a)), and the numbers for enrichment quoted herein refer to the fold enrichment or percentage of Fc-specific glycopeptides having one or more sialic acid residues, of the total Fc-specific glycopeptides. The total sialylation of an IgG molecule (including Fab and Fc sialylation) can be determined by digesting the tryptic peptides with N-Glycosidase F, followed by analysis of the released glycans (for example as described in Example 2, method (b)). In this case, the numbers refer to the number or percentage of glycans having one or more sialic acid residues of the total glycans.

A population of antibodies of the invention may be an isolated and/or purified population of antibodies, depending on the choice of antibody preparation as a starting material used for affinity chromatography.

In accordance with all embodiments of the present invention, antibodies can be extracted from blood plasma or can be produced by any one of the various procedures known in the art, e.g. as described in Harlow and Lane (1988) and (1999), loc. cit. Thus, the antibodies may, for example, be synthetically produced, and may also include peptidomimetics. Further, techniques described for the production of single chain antibodies (see, inter alia, U.S. Pat. No. 4,946,778) can be applied. Also, transgenic animals or plants (see, e.g., U.S. Pat. No. 6,080,560) may be used to express (humanized) antibodies. For the preparation of monoclonal antibodies, any technique which provides antibodies produced by continuous cell line cultures can be used. Examples for such techniques known in the art, e.g. as described in Harlow and Lane (1988) and (1999), loc. cit. and include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). It is also envisaged in the context of this invention that the term “antibody” comprises antibody constructs which may be expressed in cells, e.g. antibody constructs which may be transfected and/or transduced via, amongst others, viruses or plasmid vectors.

Because antibodies having low levels of sialylation or no sialylation in the Fab region will not be bound by the affinity chromatography in accordance with the present invention, the antibody population not bound in step (a) and collected in step (b) is a population having a decreased amount of sialylation in the Fab region of the antibodies as compared to the preparation before the affinity chromatography. In other words, this antibody population is a sialic acid depleted population. The antibody population bound in step (a) and eluted from the affinity matrix with carbohydrate is a population having an increased amount of sialylation in the Fab region of the antibodies as compared to the preparation before the affinity chromatography, due to the binding of antibodies having high levels of sialylation in the Fab region to the affinity chromatography. Thus, the antibodies of the starting population of antibodies are separated into enriched populations having either an increased or decreased amount of sialylation in the Fab region of the antibodies.

In a preferred embodiment of the method of the invention, at least one wash step is carried out between loading the column and starting elution (step c in claim 1). Suitable washing solutions are well known to the skilled person, for example, tris-buffered saline (TBS) or phosphate buffered saline (PBS) may be used.

In a more preferred embodiment of the method of the invention, the affinity chromatography has a lower affinity for sialylated Fc regions of antibodies than for sialylated Fab regions of antibodies, i.e. the affinity is selective for Fab sialylation over Fc sialylation. Thus, the affinity matrix employed in the method of the invention will preferentially bind sialic acids contained in the Fab region of the antibodies but not, or only to a smaller extent, bind sialic acids contained in the Fc region of the antibodies. Consequently, as the separation is based on Fab sialylation, the population of antibodies obtained by elution (steps d and e in claim 1) are more enriched for antibodies having a Fab sialylation than for antibodies having a Fc sialylation and, similarly, the population of antibodies obtained in step b) is more enriched for antibodies having no or almost no Fab sialylation irrespective of their Fc sialylation. Preferably, the Fc sialylation will differ by less than 100%, more preferably less than 50%, even more preferably less than 30%, most preferably less than 20%, when measuring % sialylated glycopeptides of the total glycopeptides.

The present invention further relates to a population of antibodies, wherein the population of antibodies has an altered amount of sialylation in the Fab region of the antibodies as compared to the antibody preparation prior to affinity chromatography.

Determination of whether a population of antibodies has an altered amount of sialylation in the Fab region of the antibodies as compared to the antibody preparation prior to enrichment can be carried out as described above. An altered amount of sialylation is considered any amount of sialylation that is not identical to the amount of sialylation of IVIG within the limits of detection of the plethora of methods currently available. Preferably, the amount of sialylation of the population of antibodies is at least 1.25 times higher or lower, more preferably at least 1.5 times higher or lower, more preferably at least two times higher or lower such as at least three times or at least four times higher or lower as compared to the amount of sialylation in the Fab region in the antibodies in the antibody preparation prior to enrichment. More preferably, the amount of sialylation is at least five times higher or lower as compared to the amount of sialylation in the Fab region in the antibodies in the antibody preparation prior to enrichment. As described above, the term “times higher or lower” refers to a relative increase or decrease compared to the amount of sialic acid in the antibody preparation prior to enrichment.

It is also envisaged that the amount of sialylation of the Fab region of the antibodies of the invention may be further modified.

For example, the amount of sialylation may be modified such that a further increase or decrease in sialylation in the Fab region of the antibodies is obtained, resulting in a further modification of the overall amount of sialylation of the respective population of antibodies. Both an increase as well as a decrease in the amount of sialic acid in the Fab region of the antibodies is envisaged. It is preferred that where a population of antibodies of the invention has an increased amount of sialic acid in the Fab region of the antibodies, the further modification is an additional increase in the amount of sialic acid in the Fab region of the antibodies. It is also preferred that where a population of antibodies of the invention has a decreased amount of sialic acid in the Fab region of the antibodies, the further modification is an additional decrease in the amount of sialic acid in the Fab region of the antibodies.

It is also envisaged that the pattern of sialylation, i.e. the type of sialic acid and/or their location in the Fab region of antibodies is further modified.

The modification of the sialylation pattern is effected enzymatically. The enzymatic modification may be carried out, for example, by using a sialyltransferase and a donor of sialic acid in order to increase the amount of sialic acid in the Fab region of the antibodies. Alternatively, a decrease in the amount of sialic acid in the Fab region of the antibodies may be achieved, for example, by using a sialidase enzyme, thereby removing sialic acids.

Non-limiting examples of sialyltransferases are ST3Gal III, also referred to as α-(2,3) sialyltransferase (EC 2.4.99.6), and α-(2,6) sialyltransferase (EC 2.4.99.1). α-(2,3) sialyltransferase catalyzes the transfer of sialic acid to the Gal of a Gal-β-1,3GlcNAc or Gal-β-1,4 GlcNAc glycoside (Wen et al., J. Biol. Chem. 267: 21011 (1992); Van den Eijnden et al., J. Biol. Chem. 256: 3159 (1991)) and is responsible for sialylation of asparagine-linked oligosaccharides in glycopeptides. Activity of α-(2,6) sialyltransferase results in 6-sialylated oligosaccharides, including 6-sialylated galactose. Different forms of α-(2,6) sialyltransferase exist and can be isolated from different tissues, or can be produced using recombinant techniques.

Non-limiting examples of sialidases (also referred to as neuraminidase, N-acetylneuraminate glycohydrolase) are sialidase Au, Alpha-(2-3,6,8,9) (EC 3.2.1.18); sialidase Cp, Alpha-(2-3,6) (EC3.5.1.18) and sialidase Sp Alpha-(2-3) (EC 3.5.1.18).

In order to achieve a selective modification of the amount of sialylation in the Fab region of the antibodies, the Fc region of an antibody may be protected from enzymatic activity by any of a variety of methods known in the art. For example, specific Fc-binding ligands might be employed to mask the Fc region. Non-limiting examples of such Fc-specific ligands include chemically synthesized ligands such as those described above or biological ligands such as soluble Fc receptors, Fc specific antibodies or protein A/G.

In addition, cell culture conditions may be altered to change the amount of sialylation of antibodies produced in cell culture. For example, an increased amount of sialic acid is obtained when the production rate is decreased and the osmolality is generally maintained in the range from about 250 mOsm to about 450 mOsm. This and other suitable cell culture conditions are described in, e.g., U.S. Pat. No. 6,656,466. Furthermore, it has been reported by Patel et al., (Patel et al., Biochem J, 285, 839-845 (1992)), that the content of sialic acid in antibody linked sugar side chains differs significantly if antibodies were produced as ascites or in serum-free or serum containing culture media. Moreover, it has also been shown that the use of different bioreactors for cell growth and the amount of dissolved oxygen in the medium influences the amount of galactose and sialic acid in antibody linked sugar moieties (Kunkel et al., Biotechnol. Prog., 1.6, 462-470 (2000)).

The altered amount of sialylation may be a reduction in the amount of sialylation in the Fab region, as described above. The altered amount of sialylation may alternatively be an increase in the amount of sialylation in the Fab region, as described above.

The populations of antibodies having an altered amount of sialylation in the Fab region of the antibodies may, for example, be used in the treatment of diseases such as inflammatory and autoimmune conditions, for example atherosclerosis, multiple sclerosis, SLE and others as listed above.

### DEFINITIONS

The term “antibody” as used throughout the present invention encompasses, for example, polyclonal or monoclonal antibodies. The term “antibody” also comprises derivatives or fragments thereof which still retain antigen binding specificity. Thus, also included are embodiments such as chimeric (human constant domain, non-human variable domain), single chain and humanized (human antibody with the exception of non-human CDRs) antibodies, as well as antibody fragments, like, inter alia, Fab or Fab′ fragments. Antibody fragments or derivatives further comprise Fd, F(ab′)₂, Fv or scFv fragments; see, for example, Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

The term “population of antibodies” in accordance with all embodiments of the present invention refers to a group of antibodies that is either homogeneous, i.e. comprises several molecules of only one specific antibody or is heterogeneous, i.e. comprises a plurality of different antibodies. In either case, the population of antibodies preferentially consists of or comprises antibodies of the IgG class (see also below). However, it could also comprise or consist of antibodies of the IgM or IgA class or mixtures of immunoglobulins of IgG, IgM and/or IgA class.

The term “Fab region”, as used throughout the present invention, refers to the region of an antibody composed of one constant and one variable domain from each heavy and light chain of the antibody, and which contains the sites involved in antigen binding. Each intact natural IgG antibody comprises two Fab regions. In accordance with the present invention, the term “Fab fragment” is used for those fragments of antibodies that are generally obtained when antibodies are digested with papain. Papain digestion results in the cleavage of the antibody above the disulfid bridges linking the two heavy chains and, thus, the release of the two individual Fab fragments. Digestion with pepsin, on the other hand, results in the release of the two Fab fragments linked together by the hinge region of the antibody, the so-called “F(ab′)₂ fragment”. With respect to antibodies not digested by these enzymatical digestions, the term “Fab region” refers to those parts of the antibody that would form the Fab and/or F(ab′)₂ fragments if digestion with papain or pepsin was carried out. With respect to antibody fragments obtained by papain or pepsin digestion, the Fab region corresponds to the Fab fragment. In the case of a Fab fragment, the corresponding molecule is identical to a Fab region.

However, as the glycosylation in the Fab regions is located in the variable regions of the antibodies, in the context of the present invention, Fv regions, single chain Fv fragments or other fragments containing the variable regions of the antibodies are considered equivalents.

The term “altered amount of sialylation” as used in accordance with the present invention refers to a change in the amount of sialic acid in a population of antibodies, wherein the sialic acids are located in the Fab region and/or in the Fc region of the antibodies of said population. Thus, the amount of sialylation of the enriched population of antibodies is altered if the overall amount of sialic acid in the Fab region and/or the Fc region of the antibodies of the enriched population of antibodies differs from the overall amount of sialic acid in the Fab region and/or the Fc region of the antibodies of the antibody preparation prior to enrichment. The change in the amount of sialic acid might be an increased amount of sialic acid or a decreased amount of sialic acid. Determination of whether a population of antibodies has an altered amount of sialylation in the Fab region of the antibodies as compared to the antibody preparation prior to enrichment can be carried out using any of a variety of methods known in the art. For example, the amount of sialylation can be determined for both the antibody preparation prior to enrichment and the population of antibodies of interest and the results thus obtained can directly be compared. Methods for determining the amount of sialylation are well known in the art. Non-limiting examples include the methods as detailed in the example section or pepsin digestion of the antibodies of interest, followed by separation of the resulting F(ab′)₂ and the pepsin fragments from the Fc fragment (for example using centricon) and quantification of the sialic acid in these fractions (for example with HPLC or MS or an enzyme based photometric assay, available from QA-bio LLC, Palm Desert, Ca: qa-bio.com) as described, for example, in Current Protocols in Protein Science: Unit 12.4 (Hudson et al), 12.6 (Royle et al) and 12.7 (Harvey et al), John Wiley and Sons (2006). These methods can be used for the determination of altered amounts of sialylation of all the embodiments described herein. The amount of increase or decrease in sialylation is generally expressed as % sialylated glycans of the total glycans, or for Fc sialylation % sialylated glycopeptides of total glycopeptides.

In accordance with the present invention, the term “sialic acid” refers to N- or O-substituted derivatives of neuraminic acid, a monosaccharide with a nine-carbon backbone. The most common member of this family of neuraminic acid derivatives is N-acetylneuraminic acid (Neu5Ac or NANA). A further member of the family is N-glycolylneuraminic acid (NGNA or Neu5Gc), in which the N-acetyl group of Neu5Ac is hydroxylated. Also encompassed by the term sialic acid is 2-keto-3-deoxynonulosonicacid (KDN) (Nadano et al. (1986) J. Biol. Chem. 261: 11550-11557; Kanamori et al., J. Biol. Chem. 265: 21811-21819 (1990)) as well as 9-substituted sialc acids such as a 9-O—C—C6 acyl Neu5Ac like 9-O-lactyl-Neu5Ac or 9-O-acetyl-Neu5Ac, 9-deoxy-9-fluoro-Neu5Ac and 9 azido-9-deoxy-Neu5Ac (Varki, Glycobiology 2: 25-40 (1992); Sialic Acids: Chemistry, Metabolism and Function, R. Schauer, Ed. (Springer Verlag, New York (1992)).

In accordance with the present invention, the term “immunomodulatory properties” refers to the effect the population of antibodies exerts on the immune system. An immunomodulatory effect can be immunosuppression or immunostimulation. A variety of in vitro or in vivo methods are available to the skilled person to measure the immunomodulatory properties of a substance like a population of antibodies. For example, a monocyte-derived cell line, e.g. U937 cells, can be grown and differentiated with retinoic acid or vitamin D. They can then be stimulated, e.g. with interferon-γ or phytohemagglutinin, in the presence and absence of the test substances. After appropriate incubation, the amount of inflammatory markers such as IL-8 can be measured in the supernatants of the cells, and cell surface markers on the U937 cells, e.g. CD54, CD14 or CD45, can be determined, e.g. by fluorescence activated cell analysis (FACS analysis). Substances with immunosuppressive, e.g. anti-inflammatory, properties will result in a lower amount of inflammatory markers as compared to the control samples. Substances can also be ranked for their anti-inflammatory effects with such an assay; the lower the amount of inflammatory markers produced, the higher the anti-inflammatory effect. Another example is a method using peripheral blood mononuclear cells (PBMCs) isolated from human blood, stimulated with certain substances such as loxoribine or CpG in the absence or presence of test substances. The inhibition of interferon-α production by test substances will be an indicator of an immunosuppressive, e.g. anti-inflammatory effect. Similar assays, measuring immunostimulation, are also available. For example, the same methods as described above can be used, but without using stimulation by e.g. interferon or phytohemagglutinin or similar stimulants.

The term “(blood) plasma immunoglobulin G preparation” in accordance with the present invention refers to an immunoglobulin G preparation isolated from (blood) plasma. The immunoglobulin G to be isolated from (blood) plasma may have been secreted by circulating B-cells (antibody secreting plasma cells). The (blood) plasma immunoglobulin G preparation can be obtained from a mammal such as for example human, but also by way of non-limiting example from transgenic animals expressing a human immunoglobulin G repertoire, such as for example pig, goat, sheep or cow (reviewed in Echelard Y and Meade H., “Toward a new cash cow.” Nat. Biotechnol. 2002 September; 20(9):881-2). For avoidance of doubt, “blood plasma” is often simply referred to as “plasma”—both are used interchangeably.

“Intravenous immunoglobulin G (IVIG)”, in accordance with the present invention, is well known to the skilled person and refers to a blood plasma preparation that contains the pooled IgG immunoglobulins extracted from the plasma of over one thousand blood donors. IVIG is used in medicine as an intravenously administered product for the treatment of diseases such as immune deficiencies, inflammatory and autoimmune diseases as well as acute infections. A preferred example of an IVIG is Privigen™.

“Subcutaneous immunoglobulin G (SCIG)” in accordance with the present invention, is well known to the skilled person and refers to a blood plasma preparation similar to IVIG, only that it is used in medicine as a subcutaneously administered product. A preferred example of a SCIG is Hizentra™.

A “purified population of antibodies” as used herein refers to a population of antibodies comprising only one class of antibody, such as IgGs. The population of antibodies may also be purified to the effect that it contains or essentially contains (i.e. to more than 90%) only antibodies of a specific subclass such as IgG1, IgG2, IgG3 or IgG4 or that it contains or essentially contains only antibodies of a defined specificity for an antigen. The term “isolated population of antibodies” as used herein refers to a population of antibodies that does not comprise any molecules other than the antibodies.

The term “inflammatory condition” refers to abnormalities associated with inflammation, which underlie a large number of human diseases. Inflammation is a complex biological response to harmful stimuli, and is the body's attempt to remove the harmful stimulus and initiate healing. However, abnormalities can occur, leading to inflammatory conditions, such as chronic inflammation or autoimmune diseases.

The term “autoimmune disease” refers to conditions where the immune system attacks self-antigens. Examples include rheumatoid arthritis, multiple sclerosis, Lupus, myasthenia gravis, psoriasis.

The term “neurodegenerative disease” refers to conditions where a progressive loss of structure or function of neurons is observed, including death of neurons. Examples of neurodegenerative diseases include Alzheimer's disease, Parkinson's disease, multiple sclerosis, Huntington's disease.

In accordance with the present invention, “atherosclerosis” refers to a chronic disease affecting arterial blood vessel. It is a chronic inflammatory response in the walls of arteries, in large part due to the accumulation of macrophage white blood cells and promoted by low density lipoproteins without adequate removal of fats and cholesterol from the macrophages by functional high density lipoproteins (HDL). It is caused by the formation of multiple plaques within the arteries. Inflammation is central at all stages of atherosclerosis and both innate and adaptive immuno-inflammatory mechanisms are involved. Disease progression is associated with formation of autoantibodies to oxidized lipoproteins and increase in circulating cytokines and inflammatory markers.

“Cancer”, in accordance with the present invention, refers to a class of diseases or disorders characterized by uncontrolled division of cells and the ability of these to spread, either by direct growth into adjacent tissue through invasion, or by implantation into distant sites by metastasis (where cancer cells are transported through the bloodstream or lymphatic system).

According to the present invention, an “infection” is the detrimental colonization of a host organism by a foreign species. In an infection, the infecting organism seeks to utilize the host's resources in order to multiply (usually at the expense of the host). The host's response to infection is inflammation.

Bacterial infections, in accordance with the present invention include but are not limited to Bacterial Meningitis, Cholera, Diphtheria, Listeriosis, Pertussis (Whooping Cough), Pneumococcal pneumonia, Salmonellosis, Tetanus, Typhus, Tuberculosis, Staphylococcus aureus or Urinary Tract Infections.

Viral infections, in accordance with the present invention include but are not limited to Mononucleosis, AIDS, Chickenpox, Common cold, Cytomegalovirus Infection, Dengue fever, Ebola Haemorrhagic fever, Hand-foot and mouth disease, Hepatitis, Influenza, Mumps, Poliomyelitis, Rabies, Smallpox, Viral encephalitis, Viral gastroenteritis, Viral encephalitis, Viral meningitis, Viral pneumonia or Yellow fever. Fungal infections in accordance with the present invention include but are not limited to Aspergillosis, Blastomycosis, Candidiasis, Coccidioidomycosis, Cryptococcosis, Histoplasmosis or Tinea pedis.

The figures show:

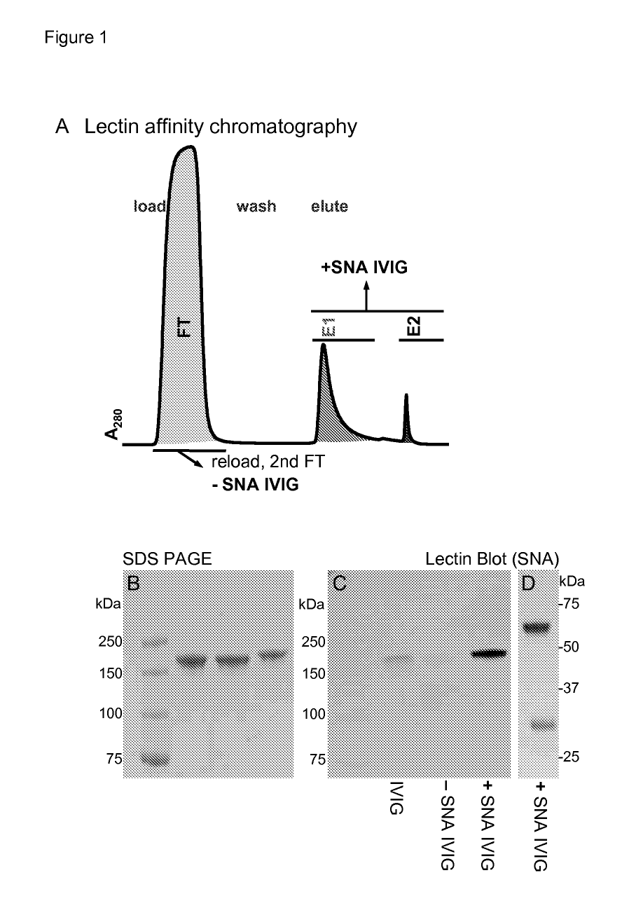

FIG. 1: Fractionation of IVIG using lectin affinity chromatography (SNA). A typical chromatogram is shown (A). Total sialic acid content in IgG was monitored with lectin blot using non-reducing conditions (B and C) or reducing conditions (D) during separation with SDS-PAGE. Shown is a Coomassie stained gel (B) and a SNA-biotin probed blot (C and D). Heavy and light chain sialylation could be detected (D).

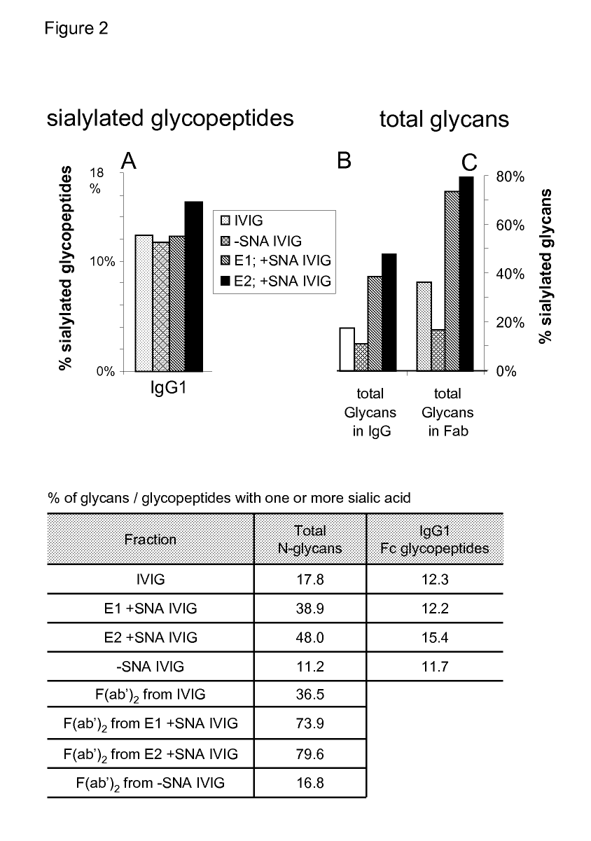

FIG. 2: Sialylated N-glycans in IVIG were measured by LC-MS analysis (as described in example 2. Relative amounts of total N-glycans, Fc-specific glycopeptides and F(ab′)₂-glycans were calculated for the different IVIG fractions. F(ab′)₂-glycans were produced from IVIG, −SA IVIG and +SA IVIG by pepsin digestion.

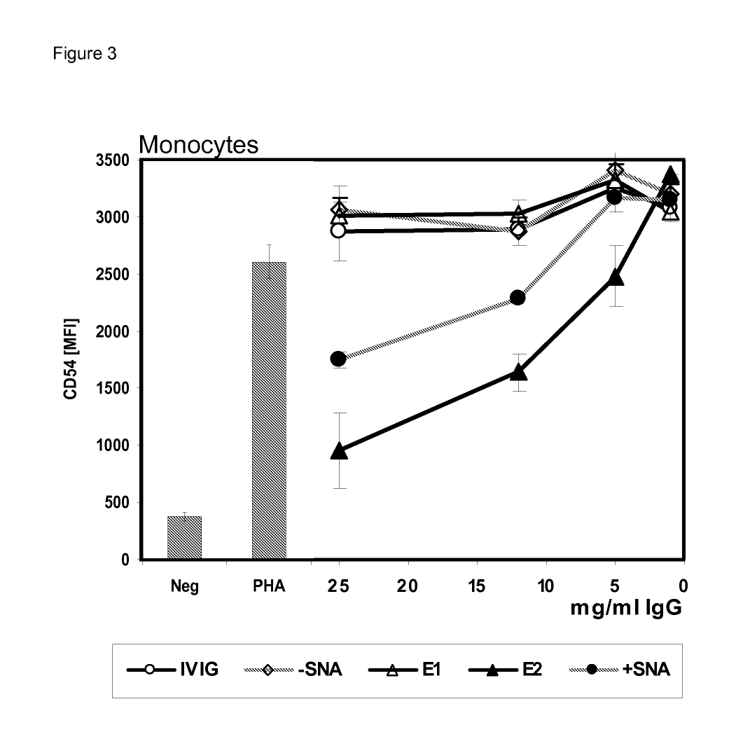

FIG. 3: Whole blood stimulated with PHA in the presence of varying concentrations of IVIg, −SNA, +SNA, E1 and E2 fractions. CD54 was measured on monocytes by flow cytometry (FACS).

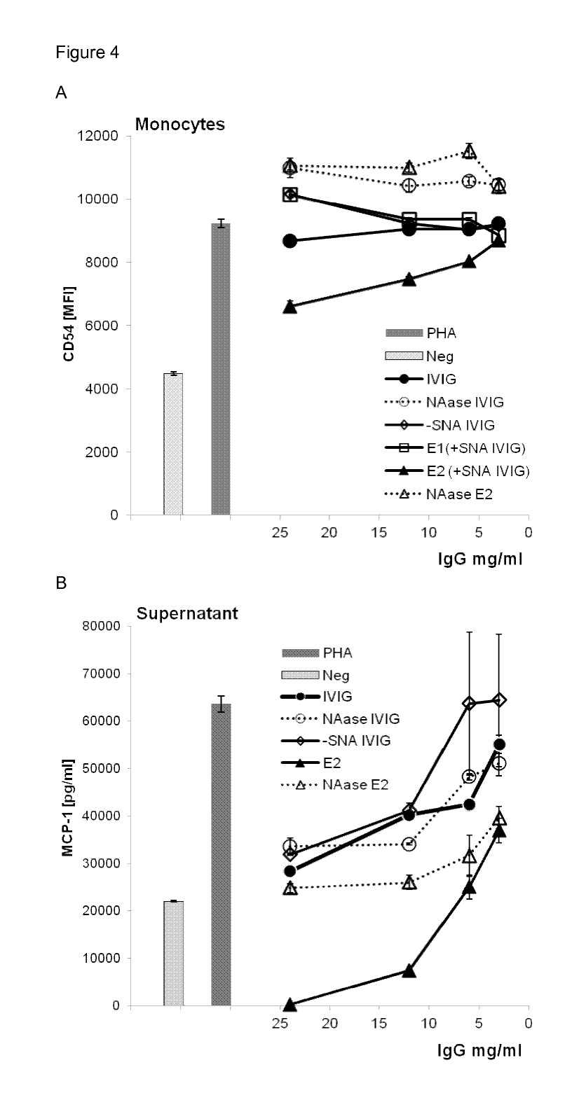

FIG. 4: Whole blood stimulated with PHA in the presence of varying concentrations of IVIg, −SNA, +SNA, E1, E2, sialidase-treated IVIg (NAase IVIg), and sialidase-treated E2 (NAase E2). CD54 was measured on monocytes by flow cytometry (FACS) (A), and MCP-1 in the supernatant was measured by ELISA (B).

### EXAMPLES

The following Examples are intended to illustrate but not to limit the invention.

### Example 1

### Fractionation of IVIG Using 2,6 Sialic Acid Specific Sambucus nigra Agglutinin (SNA)

1 g IVIG in 100 ml tris-buffered saline (TBS), 0.1 mM CaCl₂ at pH 7.5 was loaded on a 100 ml Sambucus nigra agglutinin (SNA) column. The flowthrough was collected. After washing with 300 ml of TBS containing 0.1 mM CaCl₂, the fraction bound to the SNA column (+SNA IVIG) was eluted with 200 ml of 0.5M Lactose in TBS (E1), followed with an elution with 50 ml of 0.5M lactose in 0.2M acetic acid (E2). The flowthrough fraction was run a second time over the column to obtain −SNA IVIG (FIG. 1).

Total sialic acid content in IgG was monitored with SDS PAGE (non-reducing conditions) and lectin blot (FIG. 1). The resulting IVIG fractions were separated with SDS PAGE using Nupage 10% BisTris Gels. The gels were stained with Coomassie and blotted on Nitrocellulose. The blots were probed with biotin-SNA and AP-streptavidin and visualised with chromogenic substrate. In FIG. 10, heavy and light chains were separated with SDS PAGE under reducing conditions. In lectin blots probed with SNA-biotin, both chains are clearly visible indicating light chain sialylation. Hence, significant Fab sialylation could be detected in +SNA IVIG.

### Quantification of Sialic Acid

Sialic acid was released by acidic hydrolysis and derivatization was done with 1,2-diamino-4,5-methylenedioxybenzene dihydrochloride (DMB). Quantification was performed with Reverse Phase High Performance Liquid Chromatography (RP-HPLC) using N-acetyl neuraminic acid (Neu5Ac) as a standard and expressed as sialic acid per IgG (weight/weight or Mol/Mol).

### Example 2

### Glycan Analysis

To investigate the glycosylation profile of IVIG before and after separation, tryptic glycopeptides derived from the Fc region of a typical IVIG preparation were identified and profiled and the total N-glycan population of IVIG was profiled through glycan release and analysis.

### Methods

### (a) Determination of IgG1 and IgG2/3 Fc Glycan Profile by Peptide Mapping.

IVIG samples were analyzed by liquid chromatography-mass spectrometry (LC-MS) after tryptic digestion to determine the glycan profile specific to the IgG1 and IgG2/3 Fc regions. The glycopeptide monitored for the IgG2 Fc region may also be found in the IgG3 Fc region, for this reason they are referred to as IgG2/3. Based on known relative abundance of IgG subclasses in serum, the majority of the signal seen for the IgG2/3 peptide is expected to originate from IgG2 molecules. Denaturation, reduction (heating with guanidinium-HCl and DTT) and alkylation (with Iodoacetamide) was followed by tryptic digestion using a 1:50 enzyme to substrate ratio. Tryptic peptides were isolated and purified on a C18 Solid Phase Extraction (SPE) spin column, and dried in tubes under vacuum. LC-MS analysis was performed on an Agilent Q-TOF system using an Agilent Proshell 300SB-C18 column for peptide separation. Chromatograms were extracted and integrated for each IgG1 and IgG2/3 glycopeptide, summing the [M+2H]2+ and [M+3H]3+ signals and using the calculated monoisotopic mass of the predicted structure. Data was analyzed and processed using Agilent MassHunter Software, version B.02.00 (Huddleston et al. Anal Chem 65, 877, 1993).

### Identification of IgG Glycopeptides.

LC-MS analysis of the tryptic digests resulted in complex chromatograms. In an effort to identify the potential glycopeptides, sequences of the heavy chain constant region for IgG subclasses 1 to 4 were obtained from the Entrez protein data base (accession numbers 12054072, 12054074, 12054076, and 12054078 respectively). Sequences were digested in silico. The peptides listed in Table 1 were identified as potential glycopeptides by the presence of an N-linked glycosylation consensus sequence. The theoretical weight of these peptides coupled to the common IgG glycan GOF, and the predicted [M+31-1]3+ ions were also calculated.

TABLE 1
List of predicted glycopeptides from the four IgG subclasses. N-linked
glycosylation consensus sequences are highlighted in bold. The calculated
monoisotopic masses of the bare peptide, peptide connected to the G0F glycan,
and the [M + 3H]3+ ion are given

| | | Monoisotopic | Mass with | [M + 3H]³⁺ |
|---|---|---|---|---|
| IgG | Predicted tryptic | mass | G0F | Ion |
| Subclass | glycopeptides | of peptide (Da) | glycan (Da) | (m/z) |
| 1 | EEQYNSTYR | 1186.5047 | 2633.0386 | 878.6874 |
| 2 | EEQFNSTFC*vvSVLTVVHQDWLNGK | 2935.4016 | 4379.9354 | 1460.9863 |
| 3 | EEQYNSTFR | 1172.5098 | 2617.0437 | 873.3557 |
| 4 | EEQFNSTYR | 1172.5098 | 2617.0437 | 873.3557 |

*Samples were treated with iodoacetamide during sample preparation. The calculated mass for the cysteine-containing peptide includes the mass of one carboxyamidomethyl group to account for this treatment.
** IgG Subclass 1 through 4 correspond to SEQ ID NOs. 1-4, respectively.

### (B) Determination of Global Glycan Profile

Samples were analyzed as follows to determine the global glycan profile. Dried tryptic peptides derived from 250 μg of each sample were reconstituted in 50 μL 100 mM NH₄HCO₃ and incubated with 250 U N-Glycosidase F (PNGase F) at 37° C. overnight. A second C18 SPE purification was performed to separate the deglycosylated tryptic peptides from the released glycans. The released glycans were converted to alditols using a basic sodium borohydride solution, incubating overnight at 45° C. Following decomposition of remaining borohydride, the glycan alditols were desalted in a Dowex 50 WX4-400 ion exchange resin spin column and dried. Repeated addition and evaporation of 1% acetic acid in methanol was used to remove remaining boric acid. Glycan alditols were reconstituted in starting mobile phase (10 mM NH₄HCO₃) and analyzed in duplicate by LC-MS in negative mode. Glycan separation was performed on a graphitized carbon high HPLC column using a 10 mM NH₄HCO₃/acetonitrile mobile phase system. Chromatograms were extracted and integrated for each glycan, summing the signals from the [M-1H]1-, [M-2H]2-, and [M-3H]3-signals of the glycan alditols. The preparation and analysis of the glycan alditols is similar to previously reported work (Karlsson et al. Rapid Commun Mass Spectrom 18, 2282, 2004).

### Results

As shown in FIG. 2, the sialylation level in Fc was not considerably higher in +SNA IVIG compared to IVIG, −SNA IVIG, and the E1 fraction. However, the E2 fraction showed about 2-3% higher sialylation of the IgG1 glycopeptides in the Fc region than the other antibody populations which is an increase of about 30% (FIG. 2A). On the other hand, the sialylation level of the total N-glycans, including sialylation in Fab and Fc regions, is significantly higher in the +SNA IVIG fractions E1 and E2 (FIG. 2B). As can be seen in FIG. 2C, the sialylation in the Fab regions is increased by about 100%, i.e. from 37% to 74% (E1)) and 80% (E2) of total glycans. Based on these results it can be concluded that the fractionation by the SNA affinity chromatography is mainly based on a different sialylation of the glycans in the Fab region of IVIG.

In summary, the fractionation method described in Example 1 results in IgG populations that are higher sialylated (E1 and E2 +SNA IVIG) or lower sialylated (−SNA IVIG) compared to the source IgG preparation (IVIG). The observed higher sialylation level is mainly due to a higher sialic acid content in the Fab region of IgG. The results suggest that the E2 fraction, eluted with acidic lactose after the elution of the E1 fraction with neutral lactose, may have slightly higher sialylation in both Fc and Fab regions as compared to the E1 fraction.

Glycan Analysis of F(ab′)₂ from IVIG, −SNA IVIG, E1 +SNA IVIG and E2 +SNA IVIG

F(ab′)₂ were produced by pepsin digestion of IVIG and fractions of IVIG produced as described in Example 1. IVIG was digested with pepsin (0.5 mg/g IVIG) in acetate buffer pH 4.0 for 2 hours at 37° C. The reaction was stopped by adding 2M Tris base until a pH of 8 was reached. Concentration and purification from small digestion products and buffer exchange to PBS was performed using a Centricon (30'000 Mw cut-off) (as described, for example, in Current protocols in Immunology: Unit 2.7 and 2.8 (Andrew et al) John Wiley and Sons (1997)).

Samples were analyzed by LC-MS after tryptic digestion. Denaturation, reduction, and alkylation of material was followed by tryptic digestion as described above. Samples were analyzed as described above to determine the global glycan profile (FIG. 2 C).

### Example 3

### Whole Blood Stimulation Assay

Whole blood from healthy donors was stimulated with 1 mg/l phytohemagglutinin-M (PHA). Sialic acid enhanced (+SNA; E1; E2) and depleted IVIG (−SNA) and fractions thereof have been added to the cells during the stimulation or prior to the addition of stimulating agent. In control samples whole blood without stimulation and IgG addition was incubated.

After 20+/−2 h of incubation at 37° C., the stimulation was monitored by measuring inflammatory markers in the supernatant by commercial ELISA Kits or bead array cytokine quantification Kits.

The blood cells have been washed with PBS and taken up in PBS. Inflammatory cell surface markers have been quantified by flow cytometry (FACS analysis) using fluorescent labelled antibodies (BD Pharmingen) on a Cantoll (BD Bioscience).

A dose dependent reduction of the stimulation marker (CD54) on monocytes by the E2 IVIG fraction down to the CD54 level of the negative control (blood only) was observed. The +SNA fraction showed an intermediate inhibition of the inflammatory marker, whereas −SNA (non SNA binding) and source IVIG did not show the inhibitory effect. (FIG. 3)

The experiment was repeated, including, in addition to the IgG preparations mentioned above, sialidase-treated IVIg (NAase IVIg) and sialidase-treated E2 fraction (NAase E2). Desialylation of IgG was performed by incubating IgG for 24 hours at 37° C. with 7 U/mg of neuraminidase according to the manufacturer's instructions (cloned form Clostridium perfringens, NewEngland Biolabs). Apart from measuring CD54 on monocytes by FACS, the MCP-1 concentration in the supernatant was also determined, using an ELISA kit (R&D Systems) according to the manufacturer's instructions. The results are shown in FIG. 4. Again, dose-dependent inhibitory effects were obtained as described above, with E2 showing the best inhibition of all the preparations. The inhibitory effect of E2 was completely eliminated by sialidase treatment, indicating that the sialic acid residue is important for the effect. In some of our experiments, unfractionated source IVIg also showed some inhibitory effect, albeit sometimes only at higher concentrations (e.g. at 50 mg/ml).

Similar results were obtained when the stimulation was done with LPS at a concentration of 0.1 to 1 ng/ml.

### Example 4

### The Effect of Fab Sialylation on Anti-Inflammatory Activity Tested in an in Vitro Cell Stimulation Assay

In various autoimmune and neurodegenerative diseases inflammatory conditions correlate with the disease. In an in vitro cell culture assay the inflammatory conditions observed in vivo are modeled to test for anti-inflammatory activity of different sialic acid enriched and depleted IVIG and Fab fractions.

Many Systemic Lupus Erythematosus (SLE) patients have a Type I “IFN signature” that correlates with disease activity and severity (Bengtsson et al, (2000) Lupus 9(9) 664-671). Immune complexes (ICs) containing RNA or DNA nucleoproteins can be endocytosed to stimulate Toll-like receptors (TLRs 7,9) leading to IFN-α production (Vollmer et al (2005) J. Exp. Med. 2002(11), 1575-1585). In this experiment the goal is to determine if and how sialylated (SA+) IgG modulates IFN-α production after TLR7/TLR9 stimulation of human cells.

Peripheral blood mononuclear cells (PBMCs) are isolated from heparinized venous blood of healthy volunteers by density-gradient centrifugation over Ficoll-Paque (Amersham Biosciences). PBMC cultures (5×10⁵/well in a 96-well round-bottom tissue culture plate, Nunc, total volume 150 μL) are stimulated with Loxoribine (200 μM, Invivogen) or CpG 2216 (200 nM, Invivogen) and treated with IVIG and sialic acid enriched (i.e. E1 and E2 fractions) or depleted (i.e. −SNA) IVIG as described in Example 1) or the corresponding F(ab′)2 or Fc portion at a concentration of 500 μg/ml of IgG or an equimolar amount of F(ab′)2 and Fc fragments (333 μg/ml F(ab′)2 resp. 167 μg/ml Fc). Supernatants are collected after 20±2 h, and IFN-α is quantified by an in-house ELISA using commercially available antibodies as described (Santer et al., (2009) J. Immunol. 182, 1192-1201). Results are expressed as the percentage of inhibition of IFN-α relative to cells that did not receive IVIG treatment.

E2 shows enhanced reduction of inflammatory markers compared to −SNA IVIG, IVIG and E1, as well as the total eluted +SNA fraction. It can be seen that in equimolar concentration the F(ab′)2 preparations give an about equivalent level of inhibition as their whole IgG counterparts, indicating that it is the Fab portion having an increased sialylation that is responsible for the greater IFN-alpha inhibition.

### Example 5

### The Effect of Fab Sialylation on In Vitro Anti-Inflammatory Activity, Using the Human Monocyte Cell Line U937

U937 cells (ATCC) are grown in culture according to instructions provided by ATCC. The cells are differentiated a day before the experiment with retinoic acid (0.5 μM) and/or Vitamin D.

The differentiated U937 cells are harvested by centrifugation at 400×g and re-suspended with PBS containing 1% human Albumin. The cells are then stimulated with 50 to 500 Units Interferon-gamma (IFNγ) or 0.1 to 2 microgram phytohemagglutinin (PHA). Sialic acid enhanced (i.e. E1 and E2 fractions) and depleted (i.e. −SNA) IVIG and fragments thereof are added to the cells during the stimulation or prior to the addition of the stimulating agents.

After 20+/−2 h of incubation at 37° C., the stimulation is monitored by measuring inflammatory markers in the supernatant by commercial ELISA kits or Bead Array for human inflammatory cytokines from Becton Dickinson (BD Biosciences). IL-8 and neopterin is measured using ELISA kits (Biosource/Demeditec diagnostics).

Surface markers (CD54, CD14, CD45) are quantified by FACS analysis using fluorescent labelled antibodies (BD Pharmingen) on a BD FACS Cantoll (BD Bioscience).

A reduction of inflammatory markers by the +SNA fractions of IVIG, in particular by the E2 fraction, is observed as compared to unfractionated IVIG and the −SNA fraction.

### Example 6

### Monocyte Derived Dendritic Cell (moDC) Stimulation Assay

Isolation of Human Monocytes and Differentiation of Immature Monocyte-Derived Dendritic Cells (iMoDCs)

Peripheral Blood Mononucelar Cells (PBMCs) are isolated from buffy coat (Blutspendedienst SRK, Bern, CH), containing the cellular part of the whole blood donation of one donor, by density centrifugation. The buffy coat is 1/1 diluted in PBS. 25 ml of the diluted buffy coat are loaded on 15 ml Ficoll Plaque (GE Healthcare Europe GmbH, Otelfingen, CH) and centrifuged for 35 mins (RT, 400 g, no brakes for stopping). The cell layers containing the PBMCs are collected and pooled. After washing (twice in 50 ml PBS), monocytes are isolated by magnetic cell sorting (MACS) using CD14-MACS beads (Miltenyi Biotech GmbH, Bergisch Gladbach, GER). The correct number of PBMCs (10× the number of monocytes needed) is taken up in MACS buffer (PBS, 0.5% BSA, 2 mM EDTA) and incubated with anti-CD14 mAb-coated MACS beads (80p1/10⁷ PBMCs; Miltenyi Biotech GmbH) for 20 mins on ice (dark). After washing with MACS buffer (centrifuged for 10 mins at 200 g), bead-labelled cells are loaded on a pre-washed (3 ml MACS buffer) LS MACS column (Miltenyi Biotech GmbH), washed (3× with 3 ml MACS buffer) and flushed with 5 ml MACS buffer after removing of the column from the magnetic field. After washing with PBS, the freshly isolated monocytes are taken up in RPMI-1640, containing 10% iFCS (Biochrom AG, Berlin, DE), 10'000 U/ml pencicillin and streptomycin (Amimed, BioConcept, Allschwil, CH) and 10 mM HEPES, and distributed to 24-well (0.5×10⁶ monocytes/well (ml)) or 48-well plates (0.25×10⁶ monocytes/well (0.5 ml)) (BD Biosciences) at a concentration of 0.5×10⁶ monocytes/ml. The purity of the isolated monocytes is assessed by FACS analysis by CD14 staining. Details of the FACS analysis are described later.

During the following 6 days, the monocytes are differentiated into immature monocyte-derived dendritic cells (iMoDCs) in presence of 50 ng/ml GM-CSF (R&D Systems Europe Ltd., Abingdon, UK) and 20 ng/ml recombinant human IL-4 (PeproTech EC Ltd., London UK). During differentiation, every second day, half of the medium gets removed and replaced by fully supplemented (see above) fresh medium. After 6 days, the cells are analysed for DC-phenotype by light microscopy and by FACS analysis. Cell surface expression of CD14, DC-SIGN(CD209), CD11c and CD1a (all monoclonal Abs from BD Biosciences) is assessed. Details of the FACS analysis are described later.

FACS Analysis of Freshly Isolated Monocytes, Differentiated iMoDCs and Maturated MoDCs

Approximately 60,000-100,000 cells in 50 μl FACS buffer (PBS, 2% iFCS) are stained with 1-2 μl of ready-to-use mAbs (anti-CD14, CD11c, CD1a, DC-SIGN, HLA-DR, CD80, CD86, CD83, CD64, CD32 and CD16; all Abs are directly labelled monoclonal Abs of BD Biosciences) and incubated for 30 mins at 4° C. (dark). The cells are washed with PBS and taken up in 50 μl PBS, fixed in 350 μl×BD CellFix (BD Biosciences) and stored in the dark at 4° C. until acquisition. Per sample, 10,000 cells are acquired with a FACS Calibur and analysed by the BD CellQuest software (both BD Biosciences).

Stimulation of iMoDCs by Immune Complexes

Immature (i)MoDCs are pre-incubated for 3 h with different amounts of IVIG fractions (as produced according to example 1) and fragments thereof. iMoDCs are stimulated with 0.1-100 μg/ml LPS. 0.5×10⁶ iMoDCs, differentiated in 24 well plates in fully supplemented RPMI (incl. 50 ng/ml GM-CSF & 20 ng/ml IL-4), are incubated with LPS during 24 hs at 37° C., 5% CO₂. For the analysis of IC-mediated stimulation (maturation), FACS analysis and cytokine measurements are performed. The cells are harvested by washing them away from the well bottom via pipetting. The cell suspension is collected and stored on ice immediately. After centrifugation (300 g, 10 mins, 4° C.), the supernatants are collected and stored at −20° C. for later cytokine measurement. The cells are taken up in 50 μl of FACS buffer (PBS, 2% iFCS) and FACS analyses are performed as described above. Cell surface expression of the two co-stimulatory molecules CD80 (B7-1) and CD86 (B7-2) is measured as well as the DC-specific maturation marker CD83 (all directly labelled mAbs, BD Biosciences).

### Cytokine Measurements in Cell Culture Supernatants

Cell culture supernatants of the iMoDC-stimulation experiments are collected and stored at −20° C. until cytokine measurement. IL-12p70, IL-10, IL-6, IL-8, TNF-α and IL-1β are measured according to manufacturer's instructions by a Cytometirc Bead Array (CBA) for human inflammatory cytokines from Becton Dickinson (BD Biosciences).

A reduction of inflammatory markers by the +SNA fractions of IVIG (E1 and E2) is observed, indicating the anti-inflammatory properties of the immunoglobulin enriched for increased sialylation. The effect is more pronounced with the E2 fraction. It can be seen that in equimolar concentration the F(ab′)2 preparations give an about equivalent level of inhibition as their whole IgG counterparts, indicating that it is the Fab portion having an increased sialylation that is responsible for the greater IFN-alpha inhibition.

### Example 7

### Assessment of the Beneficial Effect of Sialylated IVIg as Compared to Conventional IVIg in EAE Mouse Model

Assessment of anti-inflammatory effects of IVIG+SNA fractions can be observed in autoimmune models and the underlying mechanism can be studied. In this model, it is shown that IVIG exerts its anti-inflammatory effects through an expansion of regulatory T cells. Using EAE, a comparison among +SNA (E1 and E2) Fc, +SNA (E1 and E2) F(ab′)₂, +SNA (E1 and E2) intact IVIg and unfractionated IVIg is done. Different doses of these preparations are administered from day 0 to the peak of the disease or to day 5. Clinical scores and survival are determined.

### Induction of EAE:

C57BL/6J strains of mice are used to induce EAE. The experiments are performed in accordance with ethical rules on animal experiments. 10-12 weeks old female mice are immunized with 200 μg MOG₃₅₋₅₅ peptide (>95% purity) emulsified in CFA (1v/1v) containing 800 μg of non-viable desiccated Mycobacterium tuberculosis H37RA. A final volume of 200 μl is injected s.c. at four sites over the flanks. In addition, 300 ng of Pertussis toxin is given i.v. on the same day and two days later. Clinical signs of EAE are assessed daily by means of the following scoring system: 0—no signs; 1—tail paralysis; 2—hindlimb weakness and tail paralysis; 3—hindlimb and tail paralysis; 4—hindlimb and tail paralysis and forelimb weakness; 5—morbidum; 6—death. EAE symptoms are observed from day 10 onwards and peak around 21-25 days post-immunization with an average clinical score of 3.

A reduction of clinical symptoms by the +SNA fractions of IVIG is observed as compared to unfractionated IVIG, indicating the anti-inflammatory properties of the immunoglobulin enriched for increased sialylation. The effect of the E2 fraction is more pronounced than the effect observed with the corresponding reagents of the E1 fraction or the total eluted fraction (+SNA).

1. A method for producing an antibody population comprising:
a. subjecting an antibody preparation to affinity chromatography on Sambucus nigra agglutinin (SNA),
b. eluting antibodies bound to the SNA with a carbohydrate at neutral pH (E1 fraction), and
c. eluting remaining bound antibodies with a carbohydrate at acidic pH (E2 fraction),
wherein the E2 fraction has an enhanced immunomodulatory activity when compared to a total bound and eluted antibody fraction from the SNA (+SNA fraction).

2. The method of claim 1, wherein the carbohydrate is a sugar.

3. The method of claim 2, wherein the sugar is lactose.

4. The method of claim 1, wherein the neutral pH is a pH in the range of 6 to 8.

5. The method of claim 1, wherein the acidic pH is a pH below 5.

6. The method of claim 1, wherein the antibody preparation is an IgG preparation isolated from human plasma.

7. The method of claim 6, wherein the antibody preparation is an IgG preparation isolated from pooled human plasma from at least 1000 donors.

8. The method of claim 7, wherein the antibody preparation is an intravenous IgG (IVIG) or a subcutaneous IgG (SCIG) preparation.

9. The method of claim 1, wherein the immunomodulatory activity is determined by an in vitro assay to measure anti-inflammatory activity.

10. The method of claim 9, wherein the immunomodulatory activity of the E2 fraction is at least 10% greater than the immunomodulatory activity of the +SNA fraction or at least 10% greater than the immunomodulatory activity of the E1 fraction.

11. A population of antibodies obtained by the method of claim 1.

12. The population of antibodies of claim 11, wherein the population of antibodies comprises the E1 fraction, and wherein the E1 fraction has
a. about equivalent sialylation in the Fc region as the antibody preparation prior to affinity chromatography; and/or
b. at least 50% higher sialylation of total glycans in the Fab region than the antibody preparation prior to affinity chromatography.

13. The population of antibodies of claim 11, wherein the fraction comprises the E2 fraction, and wherein the E2 fraction has
a. at least 20% higher sialylation in the Fc region than the antibody preparation prior to affinity chromatography or than the E1 fraction; and/or
b. at least 50% higher sialylation of total glycans in the Fab region than the antibody preparation prior to affinity chromatography; and/or
c. at least 5% higher sialylation of total glycans in the Fab region than the E1 fraction.

14. A pharmaceutical composition comprising the antibody population of claim 11, and a pharmaceutically acceptable carrier or excipient.

15. (canceled)

16. A method of treating an inflammatory condition in a patient in need thereof, comprising:
administering a pharmaceutically effective amount of the antibody population of of claim 11.

17. The method of claim 16, wherein the inflammatory condition is an autoimmune disease or a neurodegenerative disease.

18. The method of claim 17, wherein the autoimmune or neurodegenerative disease is Rheumatoid arthritis, Systemic Lupus Erythematosus (SLE), Antiphospholipid syndrome, immune thrombocytopenia (ITP), Kawasaki disease, Guillain Barré syndrome (GBS), multiple sclerosis (MS), chronic inflammatory demyelinating polyneuropathy (CIDP), skin blistering diseases, Dermatomyositis, Polymyositis, Alzheimer's Disease, Parkinson's Disease, Alzheimer's Disease related to Down Syndrome, cerebral amyloid angiopathy, Dementia with Lewy bodies, Frontotemporal lobar degeneration or vascular dementia.
